(19) United States
(12) Patent Application Publication
(10) Pub. No.: US 2021/0401624 A1
(43) Pub. Date: Dec. 30, 2021

(54) LASER SURGICAL SYSTEM FOR S-CURVE INCISION

(71) Applicant: Alcon Inc., Fribourg (CH)

(72) Inventors: Zsolt Bor, San Clemente, CA (US); Imre Hegedus, Aliso Viejo, CA (US); Keith Watanabe, Irvine, CA (US); Alireza Malek Tabrizi, Irvine, CA (US)

(21) Appl. No.: 17/356,598

(22) Filed: Jun. 24, 2021

Related U.S. Application Data
(60) Provisional application No. 63/043,861, filed on Jun. 25, 2020.

(51) Int. Cl. A61F 9/008

(52) U.S. Cl. CPC A61F 9/00836; A61F 2009/00897; A61F 2009/00872

(57) ABSTRACT

A laser surgical system comprises a laser source, scanners, delivery optics, and a computer. The laser source generates a beam of femtosecond laser pulses. The scanners direct focus spots of the beam towards points of a cornea. The delivery optics focuses the focus spots at the points of the cornea. The computer creates an incision in the cornea by instructing the optics and scanners to: direct and focus the focus spots from a posterior corneal surface, through a convex curve and a concave curve, to an anterior corneal surface to form an S-curve incision with a posterior end and an anterior end. The S-curve incision has a substantially non-planar rectangular shape with a longer side that extends from the posterior end to the anterior end and defines a longer direction. A cross-section of the incision in the longer direction exhibits the convex curve and the concave curve.

### TECHNICAL FIELD

The present disclosure relates generally to laser surgical systems, and more particularly to laser surgical systems that can create incisions in an eye.

### BACKGROUND

Ophthalmic laser surgical systems generate a pulsed femtosecond laser beam and direct the laser pulses to focus spots of an ophthalmic tissue. When the beam intensity or energy density exceeds a plasma or photodisruption threshold at a focus spot, a plasma or cavitation bubble is created in the tissue. During surgery, the laser beam can be scanned along a three-dimensional scan pattern to create a layer of bubbles that form an incision.

In laser-assisted cataract surgery (LACS), a laser may be used to form particular types of incisions. For example, the laser may be used to make an incision in the cornea into which a surgical instrument may be placed to access the interior of the eye. The incision should be designed to avoid excess leakage from the incision.

### BRIEF SUMMARY

In certain embodiments, a laser surgical system comprises a laser source, scanners, delivery optics, and a computer. The laser source generates a beam of femtosecond laser pulses. The scanners direct focus spots of the beam towards points of the cornea of an eye, where the cornea has a posterior corneal surface and an anterior corneal surface. The delivery optics focuses the focus spots at the points of the cornea. The computer creates a three-dimensional incision in the cornea by instructing the optics and the scanners to: direct and focus the focus spots from the posterior corneal surface, through a convex curve and a concave curve, to the anterior corneal surface to form an S-curve incision with a posterior end and an anterior end. The S-curve incision has a substantially non-planar rectangular shape with a longer side and a shorter side. The longer side extends from the posterior end to the anterior end and defines a longer direction. A shorter direction is substantially perpendicular to the longer direction. A cross-section of the incision in the longer direction exhibits the convex curve and the concave curve. The convex curve is convex relative to the anterior corneal surface, and the concave curve is concave relative to the anterior corneal surface.

Embodiments may include none, one, some, or all of the following features:
A cross-section of the incision in the shorter direction exhibits a straight line. The straight line may have a length in the range of 2100 to 2400 μm.
A cross-section of the incision in the shorter direction exhibits an arced line. The arced line may have: an arc height in the range of up to 500 micrometers; an arc width in the range of 1000 to 5000 micrometers; and/or an arc diameter of in the range of 4 to 18 millimeters.
The convex curve has an apex height in the range of up to 20 percent of a corneal thickness between the posterior corneal surface and the anterior corneal surface.
The concave curve has an apex height in the range of up to 20 percent of a corneal thickness between the posterior corneal surface and the anterior corneal surface.
The incision towards the posterior end is substantially tangential to a posterior line that is at a first angle to a normal line of the posterior corneal surface, where the first angle is in the range of 15 to 90 degrees.
The incision towards the anterior end is substantially tangential to an anterior line that is at a second angle to a normal line of the anterior corneal surface, where the second angle is in the range of 15 to 90 degrees.
The computer creates the three-dimensional incision in the cornea by scanning the focus spots according to a raster scan or a zig-zag scan.
The computer creates the three-dimensional incision in the cornea by scanning the focus spots starting from the posterior end and ending at the anterior end.
The computer creates the three-dimensional incision in the cornea by scanning the focus spots according to depth in a z-direction defined by a propagation direction of the beam by: starting from points with the greatest z-value, closest to the posterior corneal surface; continuing through points with smaller and smaller z-values; and ending at points with the smallest z-value, closest to the anterior corneal surface.

In certain embodiments, a method of creating an incision in an eye includes generating, by a laser source, a beam of femtosecond laser pulses. Focus spots of the beam are directed, by scanners, towards points of the cornea of an eye, where the cornea has a posterior corneal surface and an anterior corneal surface. The focus spots are focused, by delivery optics, at the points of the cornea. A three-dimensional S-curve incision in the cornea is created by a computer. The S-curve incision has a substantially non-planar rectangular shape with a longer side and a shorter side. The longer side extends from an anterior end to a posterior end and defines a longer direction. A shorter direction is substantially perpendicular to the longer direction. The computer creates the S-curve incision by controlling the optics and the scanners to direct and focus the focus spots to: create the posterior end of the S-curve incision; create a portion of the S-curve incision with a convex curve and a concave curve; and create the anterior end of the S-curve incision. The convex curve is convex relative to the anterior corneal surface, and the concave curve is concave relative to the anterior corneal surface.

Embodiments may include none, one, some, or all of the following features:
The creation of the three-dimensional S-curve incision in the cornea includes scanning the focus spots according to a raster scan or a zig-zag scan.
The creation of the three-dimensional S-curve incision in the cornea includes scanning the focus spots starting from the posterior end and ending at the anterior end.
The creation of the three-dimensional S-curve incision in the cornea includes scanning the focus spots according to depth in a z-direction defined by a propagation direction of the beam by: starting from points with the greatest z-value, closest to the posterior corneal surface; continuing through points with smaller and smaller z-values; and ending at points with the smallest z-value, closest to the anterior corneal surface.
A cross-section of the incision in the shorter direction exhibits a straight line.
A cross-section of the incision in the shorter direction exhibits an arced line.

### BRIEF DESCRIPTION OF THE DRAWINGS

FIG. 1 is a block diagram of an example ophthalmic surgical laser system that performs a procedure on an eye;

FIGS. 2A and 2B illustrate examples of S-curve incisions that may be created by the system of FIG. 1;

FIG. 3 illustrates a cross-section in the longer direction of the S-curve incisions of FIGS. 2A and 2B;

FIG. 4 illustrates a cross-section in the longer direction of another example of an S-curve incision;

FIGS. 5A and 5B illustrates a cross-section in the shorter direction of the S-curve incisions of FIGS. 2A and 2B, respectively; and

FIG. 6 is a flowchart of an example of a method of creating an S-curve incision in an eye, which may be performed by the system of FIG. 1.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

In general, the present disclosure relates to an ophthalmic laser surgical system that creates an S-curve incision in the cornea of an eye, which may be used as, e.g., a primary incision for cataract surgery. The S-curve incision has a substantially non-planar rectangular ribbon shape with convex and concave curves that yield an S-curve shape. The S-curve incision may be compared with a Z-bend incision, which has bends or corners instead of curves. The S-curve shape may reduce leakage from the incision better than the Z-bend shape and may yield improved wound self-sealing.

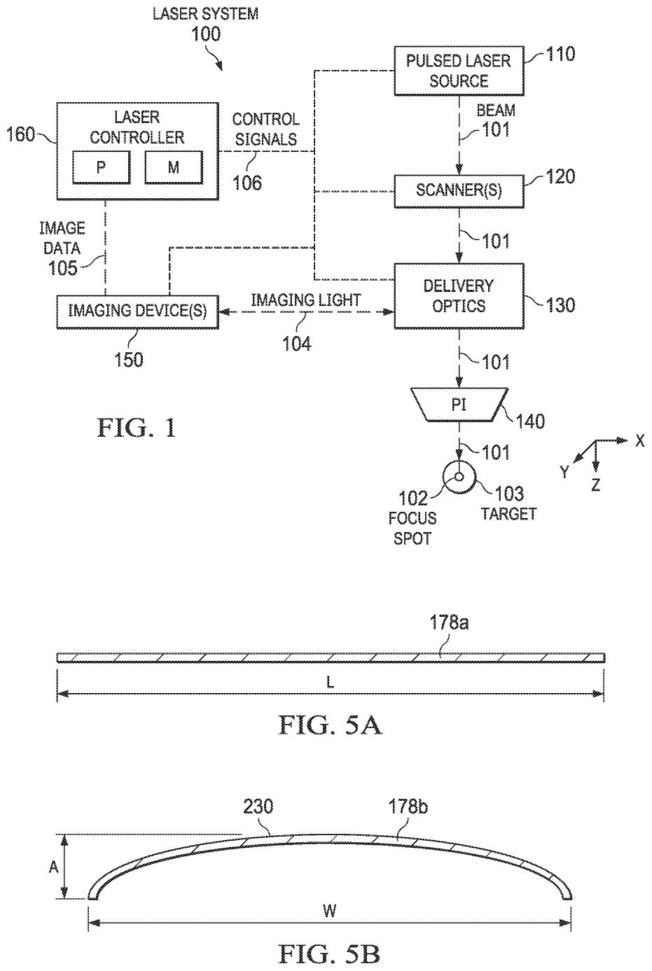

FIG. 1 is a block diagram of an example ophthalmic surgical laser system 100 that performs a procedure on a target eye 103. The system 100 includes a pulsed laser source 110, scanner(s) 120, delivery optics 130, a patient interface 140, imaging device(s) 150, and a laser controller 160 (which includes a processor P and memory M). In an example of operation, laser source 110 generates a beam 101 of femtosecond laser pulses. Scanners 120 direct focus spots of beam 101 towards points of eye 103. Delivery optics 130 focus the scanned beam 101 through patient interface 140 to focus the spots 102. During the procedure, imaging device 150 generates images of eye 103 via imaging light 104 and sends image data 105 to laser controller 160. Laser controller 160 sending instructions via control signals 106 to laser source 110, scanners 120, delivery optics 130, and/or imaging device 150 to generate a three-dimensional scan pattern of spots in eye 103. In the xyz-coordinate system of the example, the z-axis is aligned with the propagation direction of beam 101 (determined by the optical axis of laser system 100), and the xy-plane is orthogonal to z-axis.

In certain embodiments, laser source 110 comprises a laser engine capable of generating beam 101 of femtosecond laser pulses. In certain variants, laser source 110 comprises a chirped pulse amplification (CPA) laser, which may include: an oscillator to generate femtosecond seed pulses; a stretcher to stretch the seed pulses by a factor of 10-1000 to the picosecond range; an amplifier to amplify the pulses; and a compressor to compress the length of the amplified pulses back to the femtosecond range. In certain variants, laser source 110 comprises a cavity-dumped regenerative amplifier laser, which may include: an oscillator, stretcher-compressor, and optical amplifier. Examples of laser source 110 include a bulk laser, fiber laser, or hybrid laser.

In certain variants, the laser pulses generated by laser source 110 may have: a pulse duration in the range of 100 to 600, 600 to 5,000, and/or 5,000 to 10,000 femtoseconds (fs), such as 600 to 1000 fs; a per-pulse energy in the range of 0.1 to 1000 microjoule (μJ), such as 1 to 3 μJ, e.g., approximately 2 μJ; a repetition frequency in the range of 1 kilohertz (kHz) to 1 megahertz (MHz); a spot separation in the range of 0.1 to 10 micrometers (μm), such as 1 to 5 μm, e.g., approximately 3 μm; and a layer separation in the range of 0.1 to 10 micrometers (μm), such as 1 to 5 μm, e.g., approximately 2 μm. Specific laser parameters for a particular procedure may be selected based on the patient or procedure.

Scanners 120 scan beam 101 to direct focus spots 102 of beam 101 towards points of eye 103 to create an incision in eye 103 in response to instructions from laser controller 160. Scanners 120 include any suitable combination of xy-scanner(s) and z-scanner(s). An xy-scanner scans focus spot 102 of beam 101 in an xy-plane perpendicular to an optical axis of the laser system 100, while a z-scanner scans focus spot 102 of beam 101 in the z-direction along the optical axis of laser system 100. Xy-scanners and z-scanners may include steering mirror(s), galvanometer(s), lens(es), servomotor(s), etc.

Optics refers to one or more optical elements that act on (e.g., transmit, reflect, refract, diffract, collimate, condition, shape, focus, modulate, and/or otherwise act on) beam 101. Examples of optical elements include a lens, prism, mirror, diffractive optical element (DOE), holographic optical element (HOE), and a spatial light modulator (SLM). Delivery optics 130 may include a focusing objective lens, a beam expander, a birefringent lens, and other lenses to direct, collimate, condition, and/or focus the scanned beam 101 through patient interface 140 to focus spot 102 of eye 103.

Patient interface 140 may include, for example, a one or two-piece transparent applanation lens attached to a mount on delivery optics 130. The mount can provide a stable connection between the patient interface and delivery optics 130. Patient interface 140 may attach to and immobilize eye 103 during a laser procedure.

System 100 may additionally include one or more imaging devices 150. In certain embodiments, system 100 includes a surgical microscope, video microscope, digital microscope, ophthalmoscope, and/or camera to receive imaging light 104 and generate real-time images of eye 103 during a procedure. System 100 may include enhanced imaging devices to assist in guiding the laser surgery, e.g., an optical coherence tomography (OCT) imaging system to generate depth-resolved images of the inner structure of eye 103, such as the location, position, and curvature of the crystalline lens, the anterior and posterior capsules, and the cornea. Image data 105 generated by imaging device 150 may be provided to a laser controller 160.

Laser controller 160 comprises memory M storing instructions executable by a processor P to control pulsed laser source 110, scanners 120, delivery optics 130, and/or imaging devices 150. Typically, the processor of laser controller 160 comprises one or more CPUs (such as those manufactured by Intel, AMD, and others), microprocessors, field-programmable gate arrays (FPGAs), application-specific integrated circuits (ASICs), digital-signal processors (DSPs), or system-on-chip (SoC) processors communicatively coupled to memory. The memory may comprise a non-transitory computer-readable medium, and may include volatile or non-volatile memory including, magnetic media, optical media, random access memory (RAM), read-only memory (ROM), removable media, or analogous components. The memory may store software instructions executable by the processor to generate control signals 106 that control the operation of pulsed laser source 110, scanners 120, delivery optics 130, and/or imaging device 150.

In certain embodiments, laser controller 160 is a computer that generates signals 106 to control parameters of beam 101 generated by pulsed laser source 110, such as a repetition rate, pulse length, and pulse energy. Laser controller 160 may also generate signals 106 to actuate components of scanners 120 and/or delivery optics 130 to direct and focus spots 102 according to a surgical scan pattern to create an incision. Such scan patterns may be any suitable two-dimensional or three-dimensional shape or pattern, including spiral, raster, zig-zag, circular, elliptical, cylindrical, or spider patterns. Raster and zig-zag scan patterns may be described using an xy-coordinate system. The scan patterns scan along parallel lines, e.g., lines of constant y values and parallel to the x-axis. After scanning one line, the scan patterns scans to the next line in the y-direction. A raster pattern scans one line and then the next line in the same x-direction. A zig-zag pattern scans one line in one x-direction and then the next line in the opposite x-direction.

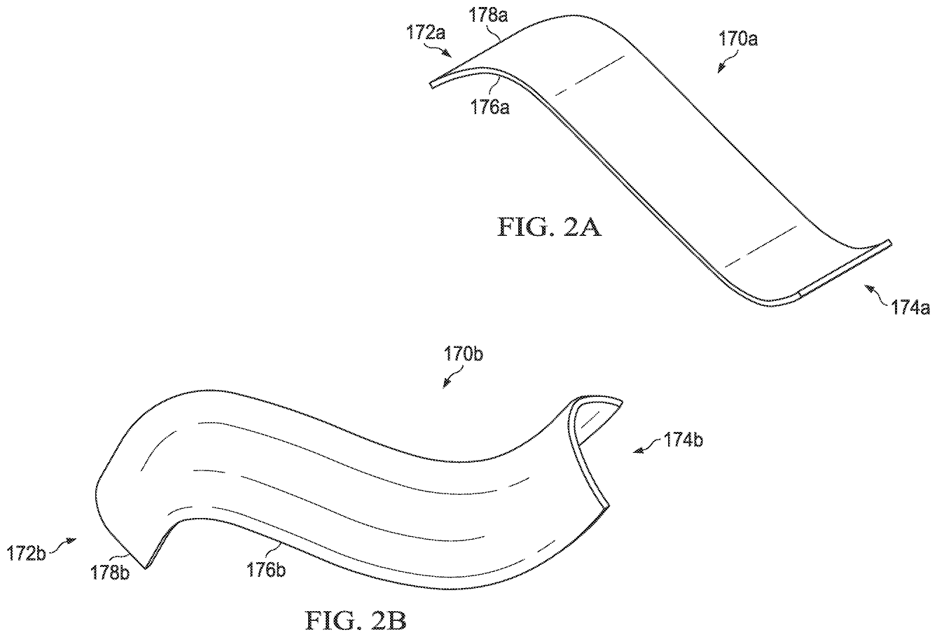

FIGS. 2A and 2B illustrate examples of S-curve incisions 170 (170a, 170b) that may be created by system 100 of FIG. 1. An incision 170 has a substantially non-planar rectangular shape with longer sides 176 (176a, 176b) and shorter sides 178 (178a, 178b). Longer sides 176 extend from a posterior end 172 (172a, 172b) to an anterior end 174 (174a, 174b) and defines a longer direction. Longer sides 176 may have any suitable length, e.g., a length in the range of 500 to 4000 μm, such as a length in the range of 500 to 1000, 1000 to 2000, 2000 to 3000, and/or 3000 to 4000 μm. Longer sides 176 are approximately the same length, but may differ in length by approximately up to 10%. Shorter sides 178 are defined by posterior end 172 and/or anterior end 174. In the example, shorter sides 178 are substantially perpendicular to longer sides 176, so a shorter direction is substantially perpendicular to the longer direction. Shorter sides 178 may have any suitable length, e.g., a length in the range of up to 1000 μm, such as a length in the range of 1 to 100, 100 to 500, and/or 500 to 1000 μm. Shorter sides 178 are approximately the same length, but may differ in length by approximately up to 10%.

The cross-section of incision 170 in the longer direction exhibits a convex curve and a concave curve. The convex curve is convex relative to anterior corneal surface, and the concave curve is concave relative to anterior corneal surface. The cross-section of incision 170a of FIG. 2A in the shorter direction is straight, and the cross-section of incision 170b of FIG. 2B in the shorter direction is arced.

As discussed above, the S-curve incision may be used as, e.g., a primary incision for cataract surgery. A primary incision may receive, e.g., a phacoemulsification handpiece tip to perform a phacoemulsification procedure or an assistance instrument, such as a pick to move cataract pieces toward the handpiece tip. The intraocular pressure (IOP) is typically elevated during the process to reduce the chances that the eye collapses. If an incision is open, the pressure falls below the natural or normal IOP level. The S-curve shape may reduce leakage from the incision better than the Z-bend shape, which may yield improved wound self-sealing. Accordingly, the S-curve may have advantages.

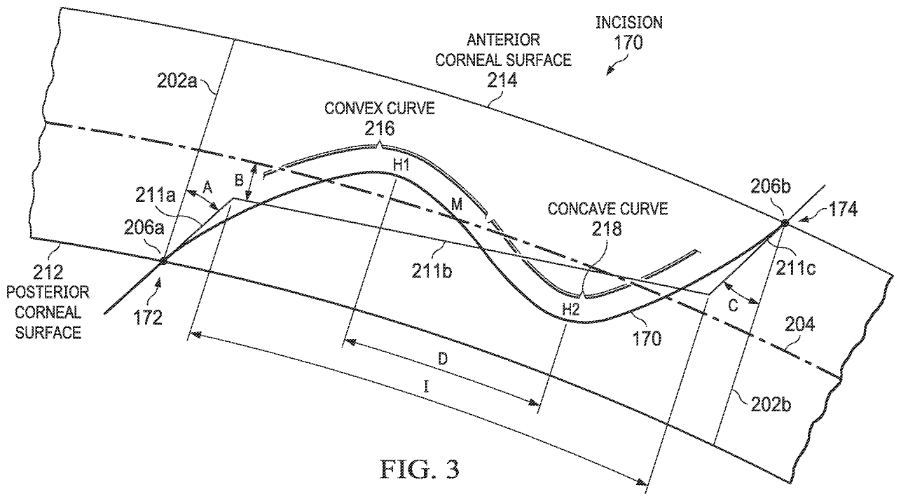

FIG. 3 illustrates a cross-section in the longer direction of S-curve incisions 170 of FIGS. 2A and 2B in a cornea with a posterior corneal surface 212 and an anterior corneal surface 214. The cross-section may be located at generally at a middle line of the incision 170 in the longer direction, i.e., along a line connecting the midpoints of shorter sides 178. However, a cross-section may be located at any suitable location of incision 170, e.g., along a longer side 176 or along a line between the middle line and a longer side 176.

In the illustrated example, normal lines 202 (202a, 202b), layer line 204, and Z-lines 211 (211a-c) are shown to aid in the description of S-curve incision 170. Normal lines 202 and layer line 204 are described relative to structures of the eye, e.g., posterior corneal surface 212 and anterior corneal surface 214. A normal line 202 may be: orthogonal to posterior corneal surface 212 and/or anterior corneal surface 214; or an average of the normal lines of posterior corneal surface 212 and anterior corneal surface 214; or the shortest distance between posterior corneal surface 212 and anterior corneal surface 214.

In the illustrated example, incision 170 intersects posterior corneal surface 212 at point 206a and anterior corneal surface 214 at point 206b. In this example, normal line 202a also intersects posterior corneal surface 212 at point 206a, and normal line 202b also intersects the point of anterior corneal surface 214 at point 206b. In another example, incision 170 might not quite reach posterior corneal surface 212 and/or anterior corneal surface 214. In this example, one or more imaginary extensions of incision 170 may be drawn to intersect posterior corneal surface 212 at point 206a and/or anterior corneal surface 214 at point 206b. Layer line 204 may be the line where the cross-section intersects a plane between posterior corneal surface 212 and anterior corneal surface 214 that is a predetermined distance away from posterior corneal surface 212 and/or anterior corneal surface 214. The distance may be specified by a length and may have any suitable value, e.g., 100 to 150, 150 to 180, 180 to 200, 200 to 220, 220 to 240, 240 to 260, and/or 260 to 300 μm from anterior corneal surface 214 (or from posterior corneal surface 212). Alternatively or additionally, the distance may be specified by a percentage of the corneal thickness and may have any suitable value, e.g., 10 to 20, 20 to 30, 30 to 35, 35 to 40, 40 to 50, 50 to 60 and/or 60 to 70 percent of corneal thickness from anterior corneal surface 214 (or from posterior corneal surface 212).

Z-lines 211 are described relative to normal lines 202. In the illustrated example, posterior Z-line 211a intersects posterior corneal surface 212 at point 206a, with an angle A between normal line 202a and Z-line 211a. Angle A may have any suitable value, e.g., a value in the range of 15 to 90 degrees, such as in the range of 15 to 30, 30 to 45, 45 to 60, 60 to 75, and/or 75 to 90 degrees. Anterior Z-line 211c intersects anterior corneal surface 214 at point 206b, with an angle C between normal line 202b and Z-line 211c. Angle C may have any suitable value, e.g., a value in the range of 15 to 90 degrees, such as in the range of 15 to 30, 30 to 45, 45 to 60, 60 to 75, and/or 75 to 90 degrees. Z-line 211b connects Z-lines 211a and 211c, with an angle B between layer line 204 and Z-line 211b. Z-line 211b has a length L and a midpoint M. Angle B may have any suitable value, e.g., a value in the range of 10 to 20, 20 to 30, 30 to 40, and/or 40 to 50 degrees.

Incision 170 extends from a posterior end 172 to an anterior end 174, which may be located at any suitable part of an eye. In the example, posterior end 172 is located at posterior corneal surface 212 and anterior end 174 is located at anterior corneal surface 214. In other examples, posterior end 172 may be posterior or anterior to posterior corneal surface 212 and/or anterior end 174 may be posterior to anterior corneal surface 214. Incision 170 is substantially tangential to Z-line 211a near posterior corneal surface 212, and is substantially tangential to Z-line 211c near anterior corneal surface 214.

Incision 170 includes convex curve 216 and concave curve 218, and intersects line 211b approximately between convex curve 216 and concave curve 218. In the illustrated example, convex curve 216 is convex relative to anterior corneal surface 214, and concave curve 218 is concave relative to anterior corneal surface 214. Of course, convexity and/or concavity may be described relative to any other suitable structure, e.g., posterior corneal surface 212. Curves 216, 218 may have any suitable dimensions. A curve 216, 218 has an apex height h (h1, h2). Apex height h is the distance between the apex of a curve and line 211b. In the example, convex curve 216 has an apex height h1, and concave curve 218 has an apex height h2. Apex height h may have any suitable value, e.g., a value in the range of up to 20 percent of the corneal thickness (i.e., the thickness between posterior corneal surface 212 and anterior corneal surface 214), e.g., a percentage in the range of 0 to 5, 5 to 8, 8 to 12, 12 to 15, and/or 15 to 20 percent of corneal thickness, such as 10 percent. Distance d is the distance between apex heights h1 and h2, and may have any suitable value, e.g., a value in the range of 30 to 70 percent (such as 30 to 40, 40 to 60, and/or 60 to 70 percent) of the length of a longer side 176.

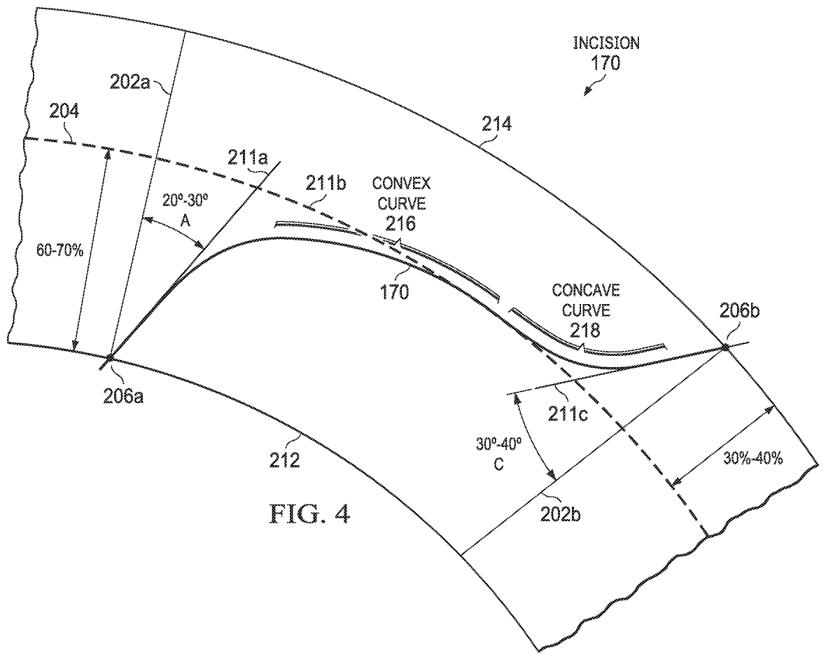

FIG. 4 illustrates a cross-section in the longer direction of another example of an S-curve incision 170. In the example, layer line 204 is a 30 to 40 percent distance away from anterior corneal surface 214. Z-line 211b substantially coincides with layer line 204. Z-line 211a is at an angle A of 20 to 30 degrees with normal line 202a, and Z-line 211c is at an angle C of 30 to 40 degrees with normal line 202b. Apex height h1 of convex curve 216 is zero, and apex height h2 of concave curve 218 is zero.

FIGS. 5A and 5B illustrates a cross-section in the shorter direction of S-curve incisions 170 of FIGS. 2A and 2B, respectively. FIG. 5A shows that the cross-section in the shorter direction of incision 170a is substantially straight with a length L. Length L may have any suitable length, e.g., in the range of 2000 to 2100, 2100 to 2200, 2200 to 2250, 2250 to 2350, 2350 to 2400, and/or 2400 to 2500 μm.

FIG. 5B shows that the cross-section in the shorter direction of incision 170b has an arc 230. Arc 230 has an arc height A, an arc width W, and an arc diameter. Arc 230 may have any suitable dimensions. In certain embodiments, arc 230 may have: an arc height H in the range of up to 500 μm, e.g., 50 to 100, 100 to 200, 200 to 250, 250 to 350, 350 to 400, and/or 400 to 500 μm, such as 300 μm; an arc width W in the range of 1000 to 5000 μm, e.g., 1000 to 2000, 2000 to 2200, 2200 to 2250, 2250 to 3000, 3000 to 4000, and/or 4000 to 5000 μm, such as 2300 μm; and an arc diameter of in the range of 4 to 18 millimeters (mm), e.g., 4 to 5, 5 to 9, 9 to 14 and/or 14 to 18 mm, such as 4.7 mm.

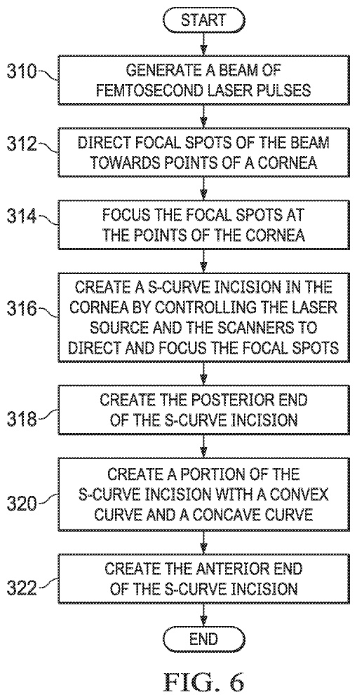

FIG. 6 is a flowchart of an example of a method of creating an S-curve incision 170 in an eye 103, which may be performed by system 100 of FIG. 1. The method starts at step 310, where laser source 110 generates a beam 101 of femtosecond laser pulses. Scanners 120 direct focus spots of the beam 101 towards points of the cornea of eye 103 at step 312. The cornea has a posterior corneal surface 212 and an anterior corneal surface 214. Delivery optics 130 focus the focus spots at the points of the cornea at step 314.

A computer (such as laser controller 160) creates S-curve incision 170 in the cornea at steps 316 to 322. S-curve incision 170 has a substantially non-planar rectangular shape with a longer side and a shorter side. The longer side extends from posterior end 172 proximate to posterior corneal surface 212 to anterior end 174 proximate to anterior corneal surface 214, and defines a longer direction. The shorter side may be the posterior end 172 and/or anterior end 174. A shorter direction is substantially perpendicular to the longer direction.

The computer creates S-curve incision 170 by controlling delivery optics 130 and scanners 120 to direct and focus the focus spots to perform the following. Posterior end 172 of S-curve incision 170 is created at step 318. A portion of S-curve incision 170 with a convex curve 216 and a concave curve 218 is created at step 320. Convex curve 216 is convex relative to anterior corneal surface 214, and concave curve 218 is concave relative to anterior corneal surface 214. Anterior end 174 of S-curve incision 170 is created at step 322.

S-curve incision 170 at steps 316 to 322 may be formed using any suitable procedure. In certain embodiments, the non-planar rectangular ribbon shape of S-curve incision 170 may be formed starting from posterior end 172 and ending at anterior end 174, where a raster scan or a zig-zag scan is used to generate the focus spots of the ribbon shape. In other embodiments, focus spots may be generated according to depth in the z-direction, where the propagation direction of beam 101 defines the +z-direction, i.e., the +z-direction is parallel to and in the same direction as beam 101. The focus spots of S-curve incision 170 are scanned starting from points with the greatest z-value, e.g., closest to posterior corneal surface 212, continuing through points with the smaller and smaller z-values, and ending at points with the smallest z-value, e.g., closest to anterior corneal surface 214. After creating the S-curve incision 170, the method ends.

A component (such as laser controller 160) of the systems and apparatuses disclosed herein may include an interface, logic, and/or memory, any of which may include hardware and/or software. An interface can receive input to the component, send output from the component, and/or process the input and/or output. Logic can perform operations of the component. Logic may include one or more electronic devices that process data, e.g., execute instructions to generate output from input. Examples of such an electronic device include a computer, processor or microprocessor (e.g., a Central Processing Unit (CPU), and computer chip. Logic may include computer software that encodes instructions capable of being executed by the electronic device to perform operations. Examples of computer software includes a computer program, an application, and an operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database and/or network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components. As another example, modifications may be made to the methods disclosed herein. The methods may include more, fewer, or other steps, and the steps may be performed in any suitable order.

To aid the Patent Office and readers in interpreting the claims, Applicants wish to note that they do not intend any of the claims or claim elements to invoke 35 U.S.C. § 112(f) unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112(f).

1. A laser surgical system comprises:
a laser source configured to generate a beam of femtosecond laser pulses;
a plurality of scanners configured to direct a plurality of focus spots of the beam towards a plurality of points of a cornea of an eye, the cornea having a posterior corneal surface and an anterior corneal surface;
delivery optics configured to focus the focus spots at the points of the cornea; and
a computer configured to create a three-dimensional incision in the cornea by instructing the optics and the scanners to:
direct and focus the focus spots from the posterior corneal surface, through a convex curve and a concave curve, to the anterior corneal surface to form an S-curve incision with a posterior end and an anterior end, the S-curve incision having a substantially non-planar rectangular shape with a longer side and a shorter side, the longer side extending from the posterior end to the anterior end and defining a longer direction, a shorter direction substantially perpendicular to the longer direction, a cross-section of the incision in the longer direction exhibiting the convex curve and the concave curve, the convex curve being convex relative to the anterior corneal surface, the concave curve being concave relative to the anterior corneal surface; wherein a cross-section of the incision in the shorter direction exhibiting a straight line.

2. (canceled)

3. The laser surgical system of claim 1, the straight line having a length in the range of 2100 to 2400 μm.

4. (canceled)

5. (canceled)

6. (canceled)

7. (canceled)

8. The laser surgical system of claim 1, the convex curve having an apex height in the range of up to 20 percent of a corneal thickness between the posterior corneal surface and the anterior corneal surface.

9. The laser surgical system of claim 1, the concave curve having an apex height in the range of up to 20 percent of a corneal thickness between the posterior corneal surface and the anterior corneal surface.

10. The laser surgical system of claim 1, the incision towards the posterior end being substantially tangential to a posterior line that is at a first angle to a normal line of the posterior corneal surface, the first angle in the range of 15 to 90 degrees.

11. The laser surgical system of claim 1, the incision towards the anterior end being substantially tangential to an anterior line that is at a second angle to a normal line of the anterior corneal surface, the second angle in the range of 15 to 90 degrees.

12. The laser surgical system of claim 1, the computer configured to create the three-dimensional incision in the cornea by scanning the focus spots according to a raster scan or a zig-zag scan.

13. The laser surgical system of claim 1, the computer configured to create the three-dimensional incision in the cornea by scanning the focus spots starting from the posterior end and ending at the anterior end.

14. The laser surgical system of claim 1, the computer configured to create the three-dimensional incision in the cornea by:
scanning the focus spots according to depth in a z-direction defined by a propagation direction of the beam by:
starting from points with the greatest z-value, closest to the posterior corneal surface;
continuing through points with smaller and smaller z-values; and
ending at points with the smallest z-value, closest to the anterior corneal surface.

15. A method of creating an incision in an eye, comprising:
generating, by a laser source, a beam of femtosecond laser pulses;
directing, by a plurality of scanners, a plurality of focus spots of the beam towards a plurality of points of a cornea of an eye, the cornea having a posterior corneal surface and an anterior corneal surface;
focusing, by delivery optics, the focus spots at the points of the cornea; and
creating, by a computer, a three-dimensional S-curve incision in the cornea, the S-curve incision having a substantially non-planar rectangular shape with a longer side and a shorter side, the longer side extending from an anterior end to a posterior end and defining a longer direction, a shorter direction substantially perpendicular to the longer direction, the S-curve incision created by controlling the optics and the scanners to direct and focus the focus spots to:
create the posterior end of the S-curve incision;
create a portion of the S-curve incision with a convex curve and a concave curve, the convex curve convex relative to the anterior corneal surface, the concave curve concave relative to the anterior corneal surface; and
create the anterior end of the S-curve incision;
wherein a cross-section of the incision in the shorter direction exhibiting a straight line.

16. The method of claim 15, the creating the three-dimensional S-curve incision in the cornea comprising:
scanning the focus spots according to a raster scan or a zig-zag scan.

17. The method of claim 15, the creating the three-dimensional S-curve incision in the cornea comprising:
scanning the focus spots starting from the posterior end and ending at the anterior end.

18. The method of claim 15, the creating the three-dimensional S-curve incision in the cornea comprising:
scanning the focus spots according to depth in a z-direction defined by a propagation direction of the beam by:
starting from points with the greatest z-value, closest to the posterior corneal surface;
continuing through points with smaller and smaller z-values; and
ending at points with the smallest z-value, closest to the anterior corneal surface.

19. (canceled)

20. (canceled)
